(19) United States
(12) Patent Application Publication
(10) Pub. No.: US 2008/0070257 A1
(43) Pub. Date: Mar. 20, 2008

(54) High throughput screening assay for histone modifying enzyme modulators

(76) Inventors: Patrick Trojer, New York, NY (US); Danny Reinberg, Warren, NJ (US)

Correspondence Address: Jane Massey Licata;Licata & Tyrrell P.C., 66 E. Main Street, Marlton, NJ, 08053, US

(21) Appl. No.: 11/854,611

(22) Filed: Sep. 13, 2007

Related U.S. Application Data
(60) Provisional application No. 60/844,344, filed on Sep. 13, 2006.

(51) Int. Cl. C12Q 1/68

(52) U.S. Cl. 435/6

(57) ABSTRACT

The present invention provides a general method for identify agents that modulate the activity of histone modifying enzymes, such as an acetylase, deacetylase, methyltransferase, demethylase, kinase, etc. The assay the of invention employs reconstituted, immobilized nucleosomes and fluorescence-based assays, such as fluorescence-based immunoassays, scintillation proximity assays, or FRET assays to determine whether the agent modulates the activity of the histone modifying enzymes.

### INTRODUCTION

This application claims benefit of priority to U.S. Provisional Patent Application Ser. No. 60/844,344, filed Sep. 13, 2006, the content of which is incorporated herein by reference in its entirety.

### BACKGROUND OF THE INVENTION

Histone modifying enzymes (HME) have been implicated in tumorigenesis. Inhibitors of histone modifying enzymes, especially histone deacetylase inhibitors, have great potential for therapeutic use as anticancer drugs. Discovery of novel compounds that selectively inhibit single HMEs is of utmost importance to improve the therapeutic arsenal to treat cancer. Conventional high throughput assays to screen for inhibitors of HMEs are based on various histone substrates that do not reflect proper physiological conditions. It is known that HMEs have different activities on different histone substrates. In a eukaryotic cell most of the nuclear histones are complexed with DNA, termed nucleosomes.

Chromatin, the organized assemblage of nuclear DNA and histone proteins, is the basis for a multitude of vital nuclear processes including regulation of transcription, replication, DNA-damage repair and progression through the cell cycle. The basic unit of chromatin is the nucleosome, consisting of an octamer of two copies each of histones H2A, H2B, H3 and H4, as well as 147 base pairs of DNA, which wraps around this histone core (Luger, et al. (1997a) Nature 389:251-260). A number of factors, including chromatin-modifying enzymes, have been identified that play an important role in maintaining the dynamic equilibrium of chromatin (Margueron, et al. (2005) Curr. Opin. Genet. Dev. 15:163-176).

The amino termini of histones (histone tails) are accessible, unstructured domains that protrude out of the nucleosomes. Histones, especially residues of the amino termini of histones H3 and H4 and the amino and carboxyl termini of histones H2A, H2B and H1, are susceptible to a variety of post-translational modifications including acetylation, methylation, phosphorylation, ribosylation and biotinylation. One type of modification, lysine methylation, is catalyzed by histone lysine methyltransferases (HKMTs). Six lysine residues of histones H3 and H4 have been identified to be the main target sites of methylation: lysines 4, 9, 27, 36, 79 of histone H3 and lysine 20 of histone H4 (Martin & Zhang (2005) Nat. Rev. Mol. Cell Biol. 6:838-849). Besides, lysine 26 on histone H1b was also shown to be methylated in vitro and in vivo (Kuzmichev, et al. (2004) Mol. Cell 14:183-193).

Histone lysine methylation is considerably different from the other types of modifications because it is regarded more stable than other histone modifications despite the recent discovery of histone lysine demethylases. Furthermore, HKMTs have a high specificity regarding a particular methylation site. For example, in higher organisms, HKMTs have been identified that only catalyze one degree of methylation on a given lysine residue. The fact that histone lysine methylation exists in three degrees provides the basis for a highly complex regulatory system. In contrast to other modifications, which can be either present or absent, histone lysine methylation can be absent or present in a mono-, di- or tri-methylated form. In principle this suggests for each residue a quadruple instead of a binary readout. Moreover, in every multicellular organism, cells acquire specific functions through a differentiation state determined by the cell-specific pattern of gene expression, which in turn is established and maintained through the differential packaging of DNA into chromatin. HKMTs play a key role in establishing and maintaining stable gene expression patterns during cellular differentiation and embryonic development, impacting on the regulation of both transcriptional activation and repression dependent on the particular site and degree of methylation. In addition, histone lysine methylation is important as it is implicated in epigenetics, the transmission of information not encoded in the DNA from parental to daughter chromatin (Trojer & Reinberg (2006) Cell 125:213-217). Therefore, the information potential of histone lysine methylation exceeds mere gene regulation.

Histone lysine methylation and HKMTs are essential for cellular integrity. Mouse knockout studies and genetic studies in flies have shown that the deletion of various HKMTs causes death during early embryonic development (Dodge, et al. (2004) Mol. Cell Biol. 24:2478-2486; O'Carroll, et al. (2001) Mol. Cell Biol. 21:4330-4336; Pasini, et al. (2004) EMBO J. 23:4061-4071; Tachibana, et al. (2002) Genes Dev. 16:1779-1791). Moreover, deletion of HKMTs in cell culture cells lead to changes of the chromatin structure and perturbs the transcriptional state of various chromatin regions (Peters, et al. (2003) Mol. Cell 12:1577-1589; Peters, et al. (2001) Cell 107:323-33), confirming the importance of HKMTs for the maintenance of proper chromatin organization.

Importantly, histone lysine methylation and HKMTs have been implicated in disease. Studies have shown global alterations of histone modifications in cancerous cells compared to the normal cellular state. For instance, histone lysine methylation patterns were found to be completely perturbed in various types of cancer. Hence, specific loss in histone H4 lysine 16 acetylation (H4K16ac) or H4 lysine 20 trimethylation (H4K20me3) have been suggested to be a common mark of human cancer (Fraga, et al. (2005) Proc. Natl. Acad. Sci. USA 102:10604-10609; Fraga & Esteller (2005) Cell Cycle 4:1377-1381). Several HKMTs have been shown to be overexpressed in cancer cells. For example EZH2 (a HKMT mediating H3K27 methylation) has been linked to invasive prostate and breast cancer (Varambally, et al. (2002) Nature 419:624-629); RIZ1 (mediating H3K9 methylation) has been identified as tumor suppressor (Canote, et al. (2002) Oncol. Rep. 9:57-60; Carling, et al. (2003) Surgery 134:932-940; Du, et al. (2001) Cancer Res. 61:8094-8099) and MLL1 (mediating H3K4 methylation) is implicated in specific types of myeloid leukaemia.

The discovery of the first HKMT (Rea, et al. (2000) Nature 406:593-599) marked the beginning of a new era in chromatin biology. Then and now detection of the HKMT activity is achieved with in vitro histone methyltransferase (HMT) assays. Four major types of substrates are used in these HMT assays: short synthetic peptides corresponding to a number of residues from the N-terminus of histone sequences comprising the target lysine residue; single recombinant histone polypeptides; histone octamers reconstituted with recombinant histone proteins; and reconstituted nucleosomes (using reconstituted octamers and specific recombinant DNA fragments). Importantly, HKMTs can have altered enzymatic activities and site specificities dependent on the substrate used in the HMT assay. For instance, PR-SET7 only catalyzes H4K20 monomethylation in the presence of nucleosomes but not octamers (Nishioka, et al. (2002b) Mol. Cell 9:1201-1213). On the contrary, SET9, a monomethylase targeting H3K4, only targets octamers (or the single H3 protein) but not nucleosomes (Nishioka, et al. (2002a) Genes Dev. 16:479-489). EZH2, which targets H3K27 in vivo (Montgomery, et al. (2005) Curr. Biol. 15:942-947), shows an activity for both H3K9 and H3K27 in vitro using octamers as substrates (Czermin, et al. (2002) Cell 111:185-196; Kuzmichev, et al. (2002) Genes Dev. 16:2893-2905). If nucleosomes are used, the activity shifts more towards H3K27. Moreover, EZH2 exhibits different IC₅₀ values in inhibitor assays depending if octamers or nucleosomes are used as a substrate.

The promiscuity of HKMTs in HMT assays in vitro further increases with the use of synthetic peptides compared to octamers/nucleosomes. This effect probably lies in the nature of this substrate: The length of the peptides is critical since the HKMT does not only recognize the target lysine but a defined number of residues N- and C-terminal of the target lysine. Therefore, the position of the target lysine within the peptide also contributes to the recognition process. Moreover, the histone N-terminal regions are highly charged. The use of mM amounts of histone peptides leads to an extraordinary and artificial accumulation of charges in the reaction mix, which potentially increases enzyme-substrate affinities and facilitates the methylation reaction. Thirdly, the structural data of HKMTs suggest that the catalytic center is in most instances shaped like a channel or cavity but is located close to the enzyme's surface. Therefore, it is more likely that a short peptide unspecifically interacts with the enzyme in comparison to the natural substrate, the nucleosome. Artificial formation of peptide-enzyme complexes positions peptide lysine residues in vicinity of the catalytic center, thereby facilitating a methylation of a lysine that might not be methylated on nucleosomes.

Analogous to HKMTs other histone modifying enzymes show different activities on different histone substrates. Importantly, it is a fact that nucleosomes are the most relevant substrates with respect to a natural chromatin environment and physiological conditions for all HMEs.

Histone modifying enzymes including histone methyltransferases have been implicated in the formation of cancer. Therefore, the discovery of compounds that selectively inhibit the activity of HMEs will improve our knowledge of the molecular function of these enzymes, assist in understanding the role of HMEs in tumorigenesis, and provide a new therapeutic approach to human cancer. Recently, inhibitors of histone deacetylases (HDACs) have been found to negatively affect tumor progression. In this regard, U.S. Patent Application No. 20050266473 teaches a method for identifying compounds that inhibit histone methyltransferases for use in treating cancer. Likewise, U.S. Patent Application No. 20050130146 teaches a method of identifying a compound which is capable of inhibiting histone deacetylase 9. Several HDAC inhibitors are currently in clinical trials, suggesting great therapeutic potential.

Therefore, there is a need in the art for a high throughput screening assay for identifying agents which modulate the activity of histone modifying enzymes in a physiologically relevant context.

### SUMMARY OF THE INVENTION

The present invention is a method for identifying an agent that modulates the post-translational modification of a histone. The method involves contacting an immobilized reconstituted nucleosome and histone modifying enzyme with a test agent, and determining via a fluorescence-based assay whether the test agent modulates the activity of the histone modifying enzyme thereby identifying an agent that modulates the post-translational modification of a histone. In certain embodiments of the present invention, the fluorescence-based assay is a fluorescence-based immunoassay, a scintillation proximity assay, or a FRET assay.

### DETAILED DESCRIPTION OF THE INVENTION

Artificial formation of histone modifying enzyme/peptide substrate complexes can position the peptide lysine residues in vicinity of the catalytic center of the enzyme, thereby facilitating methylation of a lysine that might not be methylated under in vivo conditions on nucleosomes. Therefore, the nucleosome is the most physiologically relevant substrate to be used in in vitro histone modifying assays. Reconstitution of nucleosomes can be performed using histones purified from eukaryotic cells (“native histones”) or histones expressed and purified from non-native host cells (“recombinant histones”). Native histones are problematic in certain instances since they are already decorated with a large number of post-translational modifications which potentially affects the incorporation of additional modifications during the in vitro histone modifying assay. Thus, the present invention provides a high throughput screening assay for identifying histone modifying enzyme modulators, wherein said assay is based on the relevant physiological substrate, the nucleosome. In this regard, the present invention specifically embraces the use of reconstituted nucleosomes as substrates for histone modifying enzymes. As is known in the art, a nucleosome is approximately 146-147 bp of DNA wrapped around a histone octamer composed of pairs of each of the four core histones (H2A, H2B, H3, and H4). The chromatin fiber is further compacted through the interaction of a linker histone, H1, with the DNA between the nucleosomes to form higher order chromatin structures.

Histones of use in accordance with the present invention can be from any species including human, mouse, dog, rat, pig, etc. Moreover, the octamer can be composed of histones from one species, or alternatively reconstituted with histones from more than one species, i.e., a hybrid octamer. Exemplary histone proteins are listed in Table 1.

TABLE 1

| | | GENBANK |
|---|---|---|
| Source | Histone | Accession No. |
| Homo sapiens | H2A | NP_734466 |
| | H2B | NP_733759 |
| | H3 | NP_003484 |
| | H4 | NP_003539 |
| Mus musculus | H2A | NP_835736 |
| | H2B | NP_075911 |
| | H3 | NP_032236 |
| | H4 | NP_291074 |
| Rattus norvegicus | H2A | NP_068612 |
| | H2B | NP_072173 |
| | H3 | NP_446437 |
| | H4 | NP_073177 |

Histones purified from eukaryotic cells (“native” histones) are already decorated with a large number of histone modifications which may hamper incorporation of further modification in the in vitro assays. Therefore, these native histone may be not a suitable substrate in all instances. Accordingly particular embodiments of the present invention embrace histones which are recombinantly produced using any conventional eukaryotic or prokaryotic expression system. Such systems are well-known and routinely employed in the art. Moreover, commercial sources such as INVITROGEN, CLONTECH, STRATAGENE and PROMEGA provide a variety of different vectors and host cells for producing recombinant proteins, with and without tags (e.g., glutathione-S-transferase, FLAG, His6, etc.). Advantageously, histones prepared by recombinant methodologies can be produced without any post-translational modifications on the recombinant histone proteins.

The recombinant protein thereafter is purified from contaminant soluble proteins and polypeptides using any of the following suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, SEPHADEX G-75; ligand affinity chromatography, and protein A SEPHAROSE columns to remove contaminants such as IgG. Recombinant purified histone proteins are the desirable substrates of this invention since such substrates are reproduced with invariable quality and are of higher suitability for in vitro histone modifying assays compared to native histones

In addition to recombinant production, a protein of the invention may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer, Boston, Mass.). Various fragments of a protein of the invention may be chemically-synthesized separately and combined using chemical methods to produce a full-length molecule ((He, et al. (2003) Proc. Natl. Acad. Sci. USA 100(21):12033-8; Shogren-Knaak and Peterson (2004) Methods Enzymol. 375:62-76).

Once the core histones are produced and isolated, they are mixed with a DNA molecule desirably containing nucleosome positional repeat sequences (e.g., TATAAACGCC; SEQ ID NO:1) under appropriate conditions, e.g., as disclosed herein, so that nucleosomes are reconstituted. In some embodiments, the mixture can further contain histone H1.

In particular embodiments, the reconstituted nucleosomes of the present invention are immobilized. Immobilization, for the purposes of the present invention, means that the nucleosomes are covalently or non-covalently attached to a matrix or solid support. Such solid supports include beads, microtiter plates and the like. By way of illustration, glutathione-S-transferase tagged histones can be adsorbed onto SEPHAROSE beads (Sigma Chemical, St. Louis, Mo.) or glutathione-derivatized microtiter plates to immobilize the nucleosome. Alternatively, the DNA molecule of the nucleosome can be tagged, e.g., as disclosed herein and used to immobilize the nucleosome.

Advantageously, reconstituted nucleosomes provide for unmodified, homogenous substrates for assaying histone modifying enzymes such as histone lysine methyltransferases. Likewise, reconstituted nucleosomes allow for homogenous pre-modification of substrates (e.g., chemically or enzymatically) for subsequent use in screening assays employing histone modifying enzymes which remove post-translational modifications (e.g., histone demethylases, histone deacetylases, histone deubiquitinases, etc.). Accordingly, the term “histone modifying enzyme” encompasses enzymes which add a post-translational modification to histones, as well as enzymes which remove a post-translational modification from histones. Such enzymes are well-known in the art, and enzymes from any source, e.g., human, dog, rat, mouse, pig, etc., can be used in accordance with the instant assay. By way of example, Table 2 provides a list of suitable human enzymes as well as their histone modifying activity.

TABLE 2

| Class of Histone | | | GENBANK |
|---|---|---|---|
| Modifying Enzyme | Histone Modifying | | Accession |
| (HME) | Activity | HME | No. |
| Histone | Catalyzes the | EHMT1 | Q9H9B1 |
| Methyltransferase | transfer of one to | EHMT2 | Q96KQ7 |
| (HMT) | three methyl groups | PRMT1 | Q99873 |
| | from the cofactor S- | PRMT2 | P55345 |
| | adenosyl methionine | PRMT3 | O60678 |
| | to lysine and | PRMT6 | Q96LA8 |
| | arginine residues of | PRMT8 | Q9NR22 |
| | histone proteins. | | |
| Histone Lysine | Removes methyl | LSD1 | O60341 |
| Demethylase | groups from | JHDM1D | Q6ZMT4 |
| | conserved histone | JMJD1A | Q9Y4C1 |
| | lysine residues. | JMJD2A | O75164 |
| Histone | Acetylate conserved | HAT1 | O14929 |
| Acetyltransferase | lysine amino acids | MYST1 | Q9H7Z6 |
| (HAT) | on histone proteins | MYST2 | O95251 |
| | by transferring an | MYST3 | Q92794 |
| | acetyl group from | MYST4 | Q8WYB5 |
| | acetyl CoA to lysine | | |
| | to form ε-N-acetyl | | |
| | lysine. | | |
| Histone Deacetylase | Removes acetyl | HDAC1 | Q13547 |
| (HDAC) | groups from an ε-N- | HDAC2 | Q92769 |
| | acetyl lysine amino | HDAC3 | O15379 |
| | acid on a histone. | HDAC4 | P56524 |
| | | HDAC5 | Q9UQL6 |
| | | HDAC6 | Q9UBN7 |
| | | HDAC7A | Q8WUI4 |
| | | HDAC8 | Q9BY41 |
| | | HDAC9 | Q9UKV0 |
| | | HDAC10 | Q969S8 |
| | | HDAC11 | Q96DB2 |
| | | SirT1 | Q96EB6 |
| | | SirT2 | Q8IXJ6 |
| | | SirT3 | Q9NTG7 |
| | | SirT4 | Q9Y6E7 |
| | | SirT5 | Q9NXA8 |
| | | SirT6 | Q8N6T7 |
| | | SirT7 | Q9NRC8 |
| Peptidyl Arginine | Converts arginine | PADI4 | Q9UM07 |
| Deiminase (PADI) | residues to | | |
| | citrulline in | | |
| | histones. | | |
| Peptidyl-prolyl | Catalyzes the | Fpr4 | NP_013554* |
| cis-trans isomerase | isomerization of | | |
| | histone on conserved | | |
| | proline residues. | | |

See also the HUGO gene database.
*Saccharomyces cerevisiae sequence.

Like histones, the histone modifying enzymes of the present invention can be recombinantly or chemically produced using conventional methods. Alternatively, a histone modifying enzyme for use in the instant method can be isolated from a natural source using standard methods such as column chromatography and gel electrophoresis.

Histone modifying enzymes specifically embraced by the present invention include enzymes that add or remove the following post-translational protein modifications: acetylation (see, e.g., Sterner & Berger (2000) Microbiol. Mol. Biol. Rev. 64: 435-459), methylation (see, e.g., Zhang & Reinberg (2001) Genes Dev. 15:2343-2360), phosphorylation (see, e.g., Nowak & Corces (2004) Trends Genet. 20:14-220), ubiquitination (see, e.g., Shilatifard (2006) Annu. Rev. Biochem. 75:243-269), sumoylation (Nathan, et al. (2006) Genes Dev. 20:966-976), ADP-ribosylation (see, e.g., Hassa, et al. (2006) Microbiol. Mol. Biol. Rev. 70:789-829), deimination (see, e.g., Cuthbert, et al. (2004) Cell 118:545-553; Wang, et al. (2004) Science 306:279-283), proline isomerization (see, e.g., Nelson, et al. (2006) Cell 126:905-916) or biotinylation (e.g. Kobza, et al. (2005) FEBS J. 272(16):4249-59.). By way of illustration, the instant assay can be carried out to determine the presence, absence or degree of methylation of lysines 4, 9, 27, 36, and 79 of histone H3 or lysine 20 of histone H4 in the presence of a test agent.

In carrying out the method of the present invention, a test agent is added to a point of application, such as a microtiter well, containing an immobilized reconstituted nucleosome and one or more histone modifying enzymes. Agents which can be screened in accordance with the instant assay can be rationally designed from crystal structure information or identified from a library of test agents. Test agents of a library can be synthetic or natural compounds. A library can comprise either collections of pure agents or collections of agent mixtures. Examples of pure agents include, but are not limited to, peptides, polypeptides, antibodies, oligonucleotides, carbohydrates, fatty acids, steroids, purines, pyrimidines, lipids, synthetic or semi-synthetic chemicals, and purified natural products, derivatives, structural analogs or combinations thereof. Examples of agent mixtures include, but are not limited to, extracts of prokaryotic or eukaryotic cells and tissues, as well as fermentation broths and cell or tissue culture supernatants. In the case of agent mixtures, one may not only identify those crude mixtures that possess the desired activity, but also monitor purification of the active component from the mixture for characterization and development as a therapeutic drug. In particular, the mixture so identified can be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

Agents of interest in the present invention are those with functional groups necessary for structural interaction with proteins, particularly hydrogen bonding, and typically include at least an amine, carbonyl, hydroxyl or carboxyl group. The agents often comprise cyclical carbon or heterocyclic structures and/or aromatic or polyaromatic structures substituted with one or more of the above functional groups.

Subsequent to applying the test agent to the one or more histone modifying enzyme and reconstituted nucleosome, it is determined whether the test agent modulates the activity of the one or more histone modifying enzyme using a fluorescence-based assay. As used herein, the term “fluorescent-based assay” means that the readout of the assay is based upon a fluorescent signal. While the term fluorescence is often used only for luminescence caused by ultraviolet, it is also considered to encompass other photoluminescences. Accordingly, a fluorescent-based assay provides for the detection of light emitted at wavelengths from approximately 100 to 800 nm. In one embodiment, the fluorescence-based assay is an immunoassay. In another embodiment, the fluorescence-based assay is a scintillation proximity assay. In still a further embodiment, the fluorescence-based assay is a FRET assay.

For the purposes of the present invention, a fluorescence-based immunoassay is an assay in which an antibody is used to detect the presence or absence of a histone post-translational modification, wherein antibody binding is determined fluorimetrically. For example, a modification-state-specific primary antibody (i.e., an antibody the binds to a specific post-translationally modified histone) is added to a nucleosome which has been contacted with histone modifying enzyme in the presence of a test agent; an enzyme-conjugated secondary antibody (e.g., horseradish peroxidase-conjugated) which binds to the primary antibody is subsequently added; and a fluorogenic enzyme substrate (e.g., the horseradish peroxidise substrate 3-p-hydroxyphenylpropionic acid (HPPA)) is used to determine antibody binding. See Example 6. Alternatively, either the primary or secondary antibody can be directly labelled with a fluorophore to determine antibody binding. In this regard, particular embodiments of the present invention embrace contacting the histone modifying enzyme reaction (i.e., contacted with the test agent) with a modification-state-specific primary antibody which is detectable with a fluorescent reagent (e.g., directly by being bound with a fluorophore or indirectly with a labelled secondary antibody or enzyme-conjugated secondary antibody). Illustrative modification-state-specific primary antibodies of use in accordance with the present invention are listed in Table 3.

TABLE 3

| Modification-State-Specific | |
|---|---|
| Antibody (Ab) | Antibody Specificity |
| Phospho-Histone H3 (Ser28) Ab¹ | Histone H3 only when |
| | phosphorylated at Ser28. |
| Phospho-Histone H3 (Thr11) Ab¹ | Histone H3 only when |
| | phosphorylated at Thr11 |
| Di-Methyl Histone H3 (Lys4) Ab¹ | Histone H3 only when di- |
| | methylated on Lys4 |
| Di-Methyl-Histone H3 (Lys9) Ab¹ | Histone H3 only when di- |
| | methylated on Lys9 |
| Di-Methyl-Histone H3 (Lys27) Ab¹ | Histone H3 only when di- |
| | methylated on Lys27 |
| Di-Methyl-Histone H3 (Lys36) Ab¹ | Histone H3 only when di- |
| | methylated on Lys36 |
| Di-Methyl-Histone H3 (Lys79) Ab¹ | Histone H3 only when di- |
| | methylated on Lys79 |
| Tri-Methyl-Histone H3 (Lys4) Ab¹ | Histone H3 when tri-methylated |
| | on Lys4 |
| Ac-Histone H2B (Lys5/12/15/20) | Histone H2B acetylated at Lys5, |
| Ab² | Lys12, Lys15, Lys20 |
| Ac-Histone H3 (Lys 24)-R Ab² | Histone H3 acetylated at Lys24 |
| Ac-Histone H3 (Lys9/14) Ab² | Histone H3 acetylated at Lys9 |
| | and Lys14 |
| Ac-Histone H4 (Lys 12)-R Ab² | Histone H4 acetylated at Lys12 |
| Ac-Histone H4 (Lys 5) Ab² | Histone H4 acetylated at Lys5 |
| Ac-Histone H4 (Lys 8)-R Ab² | Histone H4 acetylated at Lys8 |
| Ac-Histone H4 (Lys16) Ab² | Histone H4 acetylated at Lys16 |
| p-(Ser 11) Ac-(Lys 15) Histone | Histone H3 phosphorylated at |
| H3 Ab² | Ser11 and aceylated at Lys15 |
| p-Histone H2B (Ser 14)-R Ab² | Histone H2B phosphorylated at |
| | Ser 14 |

¹Available from Cell Signaling Technology, Inc., Danvers, MA.
²Available from Santa Cruz Biotechnology, Inc., Santa Cruz, CA.

As an alternative to a fluorescence-based immunoassay, the present invention also provides a scintillation proximity assay. For the purposes of the present invention, a scintillation proximity assay is an assay in which nucleosomes are immobilized on a surface that contains a scintillant which emits light upon exposure to a radioisotope. Examples of such surfaces include SPA beads available from GE Healthcare (Piscataway, N.J.), which are yttrium silicate or polyvinyltoluene microspheres containing scintillant; and affinity-coated FLASHPLATES (PERKINELMER, Inc., Waltham, Mass.), wherein the interior of the wells are coated with a thin-layer scintillant plastic. In accordance with this screening assay of the invention, the histone modifying enzyme reaction (i.e., immobilized nucleosome contacted with test agent in the presence of a histone modifying enzymes) is carried out using a donor substrate containing a radioisotope. Histone modification brings the radioisotope in close proximity to the surface containing the scintillant, such that light is emitted and measured using, e.g., a scintillation counter. See Example 7.

A further approach for determining whether a test agent modulates the activity of a histone modifying enzyme involves the use of FRET. As is conventional in the art, FRET is a fluorescence-based assay that employs two different fluorescent molecules (i.e., FRET donor and acceptor fluorophores) fused to two molecules of interest. For the combined FRET effect, the emission peak of the donor must overlap with the excitation peak of the acceptor. In FRET, light energy is added at the excitation frequency for the donor fluorophore, which transfers some of this energy to the acceptor, which then re-emits the light at its own emission wavelength. The net result is that the donor emits less energy than it normally would, while the acceptor emits more light energy at its excitation frequency. For use in accordance with the instant assay, it is contemplated that the DNA molecule of the nucleosome can be labeled with a FRET donor (e.g., fluorescein) and a histone modification-state-specific primary antibody can be labelled with a FRET acceptor (e.g., tetramethylrhodamine) using conventional methods. The histone modifying enzyme reaction (i.e., immobilized nucleosome contacted with test agent in the presence of a histone modifying enzymes) is carried out using an unlabelled donor substrate and histone modification is determined with the fluorescent-labelled antibody. Modification of the histone protein results in antibody binding to the nucleosome, bringing the acceptor and donor dyes within 10-100 Å. The reaction can be measured in a fluorimeter by exciting at the absorption wavelength of the donor and detecting fluorescent emission at the acceptor wavelength. See, e.g., Example 8.

Desirably, the instant assay is adapted for high throughput screening. Accordingly, the screening assay of the invention is preferably performed in any format that allows rapid preparation and processing of multiple reactions such as in, for example, multi-well plates of the 96-well variety. Stock solutions of the agents as well as assay components are prepared manually and all subsequent pipetting, diluting, mixing, washing, incubating, sample readout and data collecting is done using commercially available robotic pipetting equipment, automated work stations, and analytical instruments for detecting the output of the assay.

In addition to the reagents provided above, a variety of other reagents can be included in the screening assays of the invention. In particular, donor substrates or cofactors can be included as sources of modifying groups for transfer to histones. In some embodiments, the modifying groups (e.g., methyl groups) are labelled with a fluorescent reagent or radioisotope. Other reagents which can be added to the reactions include salts, neutral proteins, e.g., albumin, detergents, etc. Also, reagents that otherwise improve the efficiency of the assay, such as protease inhibitors, nuclease inhibitors, anti-microbial agents, and the like can be used.

It is contemplated that agents identified in accordance with the present assay can either activate or inhibit the activity of a histone modifying enzymes. Accordingly, the term “modulating” or “modulates” is intended to encompass both activators and inhibitors. Given their use in the treatment of diseases such as cancer, particular embodiments of the present invention embrace agents that inhibit the activity of, e.g., histone deacetylases.

An agent identified in accordance with the instant assay method can be formulated into a pharmaceutically acceptable composition for therapeutic use, e.g., in the treatment of cancer. The agent can be formulated with any suitable pharmaceutically acceptable carrier or excipient, such as buffered saline; a polyol (e.g., glycerol, propylene glycol, liquid polyethylene glycol and the like); carbohydrates such as glucose, mannose, sucrose or dextrans, mannitol; amino acids such as glycine; antioxidants; chelating agents such as EDTA or glutathione; preservatives or suitable mixtures thereof. In addition, a pharmaceutically acceptable carrier can include any solvent, dispersion medium, and the like which may be appropriate for a desired route of administration of the composition. The use of sustained-release delivery systems such as those disclosed by Silvestry, et al. ((1998) Eur. Heart J. 19 Suppl. I:I8-14) and Langtry, et al. ((1997) Drugs 53(5):867-84), for example, are also contemplated. The use of such carriers for pharmaceutically active substances is known in the art. Suitable carriers and their formulation are described, for example, in Remington: The Science and Practice of Pharmacy, Alfonso R. Gennaro, editor, 20th ed. Lippincott Williams & Wilkins: Philadelphia, Pa., 2000.

The key features of the instant assay include the use of physiologically relevant histone substrates, namely nucleosomes; immobilization of nucleosomes to facilitate washes and quantification; the use of fluorescence versus radiolabelling for detecting histone modifying enzyme activity; the use of microtiter plates to carry out both the enzymatic reaction and the quantification of modified substrate; and ease of adaptability to test the entire range of histone modifying enzymes (e.g., histone acetyltransferases, histone kinases, etc.). The instant high throughput screening assay offers distinct advantages over conventional assays. While conventional approaches of screening for novel histone modifying enzyme inhibitors have employed recombinant histone modifying enzymes mixed with radiolabelled donor substrates and various histone substrates in the presence of small molecule inhibitors, these assays are suboptimal. Such assays use peptides (see, e.g., Greiner, et al. (2005) Nat. Chem. Biol. 1:143-145) which are not suitable substrates for the wide array of histone modifying enzymes. In contrast, the instant assay provides the histones in a physiologically relevant context, i.e., in nucleosomes thereby increasing the specificity of the histone modifying enzyme and the inhibitor. Indeed, it is contemplated that the IC₅₀ values of a given compound for a specific histone modifying enzyme will deviate if nucleosomes instead of histone peptides are used.

Furthermore, conventional histone modifying enzyme assays involve the use of radioactivity (³H or ¹⁴C) for reasons of sensitivity thereby posing a hazard to the technician and creating radioactive waste. Moreover, the detection of histone modifying enzyme activity in such assays requires spotting a fraction of the reaction mixture on filter paper, wherein the filters need to be washed and subjected to scintillation counting. These additional steps are time-consuming and generate additional radioactive waste. In addition, the filter-binding assay is prone to false-positive and -negative results. It depends on the washing conditions whether the entire modified protein species and/or free radiolabelled donor-substrate are bound to the filter paper. Finally, filter binding histone modifying enzyme assays show a relatively high background with respect to filter-bound radioactivity. Therefore, such assays are not suitable to screen enzymes with low in vitro histone modifying enzyme activity. In contrast, it is believed that the use of fluorescent-labelled donors or other fluorometric techniques advantageously eliminates such hazards and drawbacks associated with radioactive-based assays.

The invention is described in greater detail by the following non-limiting examples.

### EXAMPLE 1

### Production of Recombinant Histone Proteins and Amplification of Specific DNA Templates

The production of recombinant histones is performed as described previously (Luger, et al. (1997b) J. Mol. Biol. 272:301-311). Optionally, the histone sequences can be expressed as a fusion-protein with a commonly used affinity tag (e.g., FLAG, haemaglutinin, hexahistidine). A DNA (template containing nucleosome positioning sites (e.g., the plasmid pG5E4 containing nucleosome positioning sites from sea urchin 5S rDNA (Utley, et al. (1998) Nature 394:498-502)) is used for nucleosome assembly. Commonly used DNA purification procedures (e.g., Maxi-prep kit; QIAGEN) are used for the purification of plasmid DNA.

### EXAMPLE 2

### Attach Affinity Tag to Amplified DNA Templates

An affinity tag (e.g., biotin) is attached to a DNA template containing nucleosome-positioning sites. Although various DNA templates can be used the following procedure describes the preparation of the plasmid pG5E4 which contains ten 5S rDNA sequences for nucleosome positioning. Briefly, plasmid DNA is linearized using the restriction enzyme KpnI and the single strand overhangs are filled in with biotin-labeled dCTP using Klenow polymerase. Subsequently, the linearized plasmid is digested with XbaI, which releases a DNA fragment containing a biotin-tag on one end and five nucleosome positioning sites on the opposite end. This fragment can be purified using a number of commercially available methods and used for reconstitution of nucleosomes.

### EXAMPLE 3

### Reconstitution of Recombinant Octamers and Recombinant Nucleosomes

For the assembly of octamers, 2 mg of each E. coli purified, recombinant histone polypeptide is adjusted in a final volume of 8 ml (1 mg/ml) unfolding buffer (20 mM Tris-HCl, pH 7.5, 7 M guanidine hydrochloride, 10 mM DTT). The mixture is then dialyzed against refolding buffer (2 M NaCl, 10 mM Tris-HCl, pH 7.5, 1 mM EDTA, 5 mM 2-mercaptoethanol) for a minimum of 6 hours with at least three buffer changes. After the mixture is concentrated with a spin-concentrator (MILLIPORE) into less than 0.5 ml, the mixture is loaded onto a SUPERDEX-200 10/30 gel-filtration column (GE Healthcare) equilibrated with refolding buffer. Fractions are analyzed by SDS-PAGE and subsequent CBB-staining and octamer-containing fractions are pooled. Generally, 2.5 to 3 mg of purified octamer is obtained.

For the assembly of oligonucleosomes, pre-assembled octamers and affinity-tagged DNA fragments (e.g., biotinylated) that contain the nucleosome positional repeat sequence are mixed in a TE buffer (10 mM Tris-HCl, pH7.5, 0.5 mM EDTA) containing 2M NaCl and 10 mM DTT. The volume is adjusted by the addition of TE buffer to yield the following final concentrations: 0.2 mg/ml octamers and 0.2 mg/ml DNA. After a series of dialysis steps in which the buffer salt concentration is gradually reduced (1.6, 1.4, 1.2, 1.0, 0.8, 0.6, 0.01 M NaCl), the sample is concentrated to 0.3 ml with a spin-concentrator (MILLIPORE). The sample is then loaded onto a 3.5-ml CL-4B (Amersham Pharmacia Biotech) gel filtration column (5 mm×5 mm×18 cm; the void volume is exactly 35% of the column volume) equilibrated with TE buffer. Assembled oligonucleosomes elute between 1.23 and 2.5 ml, and the peak fraction is used for histone modifying enzyme assays.

### EXAMPLE 4

### Coating of Microtiter Plate Wells

Depending on the affinity tag attached to the DNA template (or optionally to the histone octamers), the wells of microtiter plates can be coated with a substance that shows high selectivity for binding to such affinity tags. By way of illustration, streptavidin-coating is disclosed herein. However, it is contemplated that any method or affinity pair can be employed. For example an antibody-antigen pair would also be suitable, as would glutathione-S-transferase and glutathione.

Streptavidin forms homotetramers and due to its biochemical properties serves as a good solid phase for binding molecules. When using streptavidin-coated microtiter plates for binding molecules, the orientation of the binding can be controlled, and even small molecules otherwise difficult to bind can be attached on a streptavidin-coated surface. Streptavidin-coated microtiter plates are commercially available and widely used for high throughput screening assays.

### EXAMPLE 5

### Histone Modifying Enzyme High Throughput Assay Using Microtiter Plates

Mammalian HMEs (e.g., HATS, HKMTS, histone kinases) are produced and purified as recombinant affinity-tagged proteins using a bacterial or baculovirus expression system. As an example, the instant assay is described for lysine methylation of histones using recombinant HKMTs. The HKMT inhibitor assay is carried out as follows: In a final reaction volume of 25 μl, 50-100 ng recombinant HKMT protein is incubated at 30° C. for 60 minutes in a reaction buffer containing 50 mM Tris-HCl, pH 8.5, 5 MM MgCl₂, 4 mM DTT, 1 μM [³H]-labeled S-adenosyl-L-methionine ([³H] SAM, 82.0 Ci/mmol, 1.0 mCi/ml, Amersham Pharmacia Biotech) and 2 μg of affinity-tagged (biotinylated) oligonucleosomes. Alternatively, unlabeled instead of [³H]-labelled SAM can be used if fluorometric quantification of enzymatic activity is preferred. For other HMEs the assay conditions are adapted to employ known donor substance and incubation conditions.

The high affinity between streptavidin and biotin allows nucleosomes to be immobilized in the microtiter-plate wells. However, the immobilized nucleosomes still serve as a substrate for the HMEs present in the reaction. To stop the reaction the supernatant is removed and the wells are washed with buffer (20 mM Tris, pH 7.9, 0.2 mM EDTA, 200 mM NaCl). Modified nucleosomes remain in the wells and the incorporated modification can be quantified as described herein. Small molecule compound libraries can be easily tested by simply adding the compounds to the HME assay reaction mix. All assays are performed in triplicate and the average of three experiments is used for the calculation of IC₅₀ values.

### EXAMPLE 6

### Detection and Evaluation of HME Assay Data

By way of illustrating the instant assay, the quantification of methyl-incorporation into nucleosomes is achieved either by scintillation counting or by fluorometric reading. For example, if [³H]-donor substrates are used, immobilized nucleosomes become radiolabelled. After washing the wells of microtiter plate to remove free [³H]-SAM and unbound proteins (like the recombinant HMEs), nucleosomes are released from the wells by competition with free biotin. Eluted nucleosomes are subjected directly to scintillation counting. If unlabeled donor substrates are used, immobilized modified nucleosomes are subject to immunodetection. First, wells are incubated with a modification-state-specific primary antibody. For instance, if the H4K20-specific monomethylase PR-SET7 is used for the HMT assay, wells are treated with anti-monomethyl-H4K20 antibody. In a second step the wells are incubated with a secondary antibody that is conjugated to horseradish peroxidase (HRP) and recognizes the primary antibody. In a third step the wells are incubated with a fluorogenic substrate (e.g., 3-p-hydroxyphenylpropionic acid (HPPA)) of HRP, which has been used previously for automated microplate fluorometric enzyme immunoassay (Tuuminen, et al. (1991a) J. Immunoassay 12:29-46; Tuuminen, et al. (1991b) Clin. Chim. Acta 202:167-177).

### EXAMPLE 7

### Homogenous Format HME High Throughput Assay

For high throughput screening it is desirable to minimize the number of steps. In the homogeneous format assay reagents are mixed and the reaction is detected and measured without additional steps. In this method, the biotin-labeled nucleosomes are immobilized on commercially available scintillation proximity assay (SPA) beads (GE Healthcare) instead of a well of a microtiter plate. The HME reaction is carried out as above using [³H]-donor substance. Histone modification brings the radioisotope [³H] in close proximity to the SPA bead containing a scintillant that emits blue light. Emitted light is measured using a scintillation counter. An alternative is to use affinity-coated FLASHPLATES (PERKIN ELMER, Inc.). The interior of these plates is coated with a thin-layer scintillant plastic. Light is also emitted by the close proximity principle.

### EXAMPLE 8

### Homogeneous Assay Using Fluorescence Resonance Energy Transfer (FRET)

This homogenous assay is carried out in solution using a donor-acceptor fluorescent pair. Two examples of these are: Fluorescein/Tetramethylrhodamine, ALEXA FLUOR 350/Alexa Fluor 488. The DNA fragment containing the nucleosome-positioning sites is labelled with Fluorescein (the FRET donor) instead of biotin. A histone modification-state-specific monoclonal antibody is labelled with Tetramethylrhodamine using standard protein chemistry. The HME assay is carried out as described in herein using unlabelled donor substrates and in the presence of the fluorescent-labelled antibody. Modification of the histone protein results in antibody binding to the nucleosome, bringing the acceptor and donor dyes within 10-100 Å. The reaction is measured in a fluorimeter by exciting at the absorption wavelength of the donor and detecting fluorescent emission at the acceptor wavelength.

What is claimed is:

1. A method for identifying an agent that modulates the post-translational modification of a histone comprising
contacting an immobilized reconstituted nucleosome and histone modifying enzyme with a test agent, and
determining via a fluorescence-based assay whether the test agent modulates the activity of the histone modifying enzyme thereby identifying an agent that modulates the post-translational modification of a histone.

2. The method of claim 1, wherein the fluorescence-based assay is a fluorescence-based immunoassay.

3. The method of claim 1, wherein the fluorescence-based assay is a scintillation proximity assay.

4. The method of claim 1, wherein the fluorescence-based assay is a FRET assay.
